(19) United States
(12) Patent Application Publication
(10) Pub. No.: US 2026/0198777 A1
(43) Pub. Date: Jul. 16, 2026

(54) NORMALIZATION OF RETINAL BIREFRINGENCE SCANNING SIGNALS

(71) Applicant: THE JOHNS HOPKINS UNIVERSITY, Baltimore, MD (US)

(72) Inventors: Boris GRAMATIKOV, Baltimore, MD (US); David Lee GUYTON, Baltimore, MD (US)

(21) Appl. No.: 19/135,958

(22) PCT Filed: Oct. 25, 2023
(86) PCT No.: PCT/US2023/035856

§ 371 Date: Jun. 5, 2025

Related U.S. Application Data
(60) Provisional application No. 63/386,974, filed on Dec. 12, 2022.

(51) Int. Cl. A61B 3/18; A61B 3/08; A61B 3/15

(52) U.S. Cl. CPC A61B 3/18; A61B 3/085; A61B 3/152

(57) ABSTRACT

Techniques for determining an alignment status of a subject's eyes are presented. The techniques utilize an optical scanner, a left polarizing beam splitter, a first left optical sensor, a second left optical sensor, a right polarizing beam splitter, a first right optical sensor, a second right optical sensor, and an electronic processor. The techniques include: normalizing a left eye electrical signal based on a power of a left eye normalizing signal at a spectrum that is characteristic of central fixation, and normalizing a right eye electrical signal based on a power of a right eye normalizing signal at a spectrum that is characteristic of central fixation. The techniques also include determining an alignment status of the subject's eyes based on a level of the normalized left eye electrical signal and a level of the normalized right eye electrical signal, and outputting an alignment status of the subject's eyes.

### CROSS-REFERENCE TO RELATED APPLICATIONS

This application is the national stage entry of International Patent Application No. PCT/US2023/035856, filed on Oct. 25, 2023, and published as WO 2024/129189 A1 on Jun. 20, 2024, which claims the benefit of U.S. Provisional Patent Application No. 63/386,974, filed on Dec. 12, 2022, which are hereby incorporated by reference herein in their entireties.

### FIELD

This disclosure relates generally to ophthalmology.

### BACKGROUND

Retinal birefringence scanning (RBS) is a method of obtaining two-dimensional information from the retina based on its birefringence, its property to change the polarization state of light upon passage through it. According to RBS, a scanning beam of polarized, e.g., near-infrared, light is reflected back from the retinal pigment epithelium, double-passing the retina. The return light (about 1/5000 of the light that entered the eye), is converted to an electrical signal and is digitized. A full or partial measurement of one or more components of the four-component Stokes vector S=[S₀, S₁, S₂, S₃] is carried out. The Stokes vector characterizes the state of polarization of the reflected light. Its first component, S₀, represents the total intensity, whereas S₁ represents the difference between a first polarization component (e.g., vertical polarization), and a second polarization component (e.g., horizonal polarization). A polarizing beam splitter interface may be used to separate the first and second polarization components, with one component being reflected and the other component being transmitted. The component that is reflected is polarized perpendicular to the plane of incidence of the light upon the beam splitter (typically denoted as “s” polarization from the German senkrecht), and the component that is transmitted is polarized parallel to the plane of incidence (typically denoted as “p” polarization for parallel). For some applications, it may be sufficient to use just S₁ to measure the change in polarization caused by the retinal birefringence. There are two areas of the retina that exhibit significant birefringence, which are of particular interest—the fovea (the most sensitive part of the retina), and the area around the optic nerve head.

RBS may be used to detect central fixation (CF). With it, binocular eye alignment is declared when both eyes (Right Eye, RE, and Left Eye, LE) are fixating at the same time on a presented target.

### SUMMARY

According to various embodiments, a system for determining an alignment status of a subject's eyes is presented. The system includes: an optical scanner disposed to direct polarized light to a left retina of the subject and to a right retina of the subject; a left polarizing beam splitter disposed to separate polarized light reflected from the left retina into a first left optical signal component and a second left optical signal component; a first left optical sensor disposed to receive the first left optical signal component and generate a corresponding first left eye electrical signal; a second left optical sensor disposed to receive the second left optical signal component and generate a corresponding second left eye electrical signal; a right polarizing beam splitter disposed to separate polarized light reflected from the right retina into a first right optical signal component and a second right optical signal component; a first right optical sensor disposed to receive the first right optical signal component and generate a corresponding first right eye electrical signal; a second right optical sensor disposed to receive the second right optical signal component and generate a corresponding second right eye electrical signal; an electronic processor; and persistent electronic memory including instructions that, when executed by the electronic processor, configure the electronic processor to perform actions including: normalizing a left eye electrical signal derived from the first left eye electrical signal and the second left eye electrical signal, where the normalizing the left eye electrical signal is based on a power of a left eye normalizing signal at a spectrum that is characteristic of central fixation, where a normalized left eye electrical signal is produced; normalizing a right eye electrical signal derived from the first right eye electrical signal and the second right eye electrical signal, where the normalizing the right eye electrical signal is based on a power of a right eye normalizing signal at a spectrum that is characteristic of central fixation, where a normalized right eye electrical signal is produced; determining an alignment status of the subject's eyes based on a level of the normalized left eye electrical signal and a level of the normalized right eye electrical signal; and outputting an alignment status of the subject's eyes.

Various optional features of the above embodiments include the following. The system may include a beam splitter disposed to direct a portion of the polarized light reflected from the left retina and the polarized light reflected from the right retina to a normalizing optical sensor, where the normalizing optical sensor provides the left eye normalizing signal and the right eye normalizing signal. The system can include a left eye beam splitter disposed to direct a portion of the polarized light reflected from the left retina to a left normalizing optical sensor, where the left normalizing optical sensor provides the left eye normalizing signal; and a right eye beam splitter disposed to direct a portion of the polarized light reflected from the right retina to a right normalizing optical sensor, where the right normalizing optical sensor provides the right eye normalizing signal. The system can include a first left amplifier coupled to an output of the first left optical sensor; a second left amplifier coupled to an output of the second left optical sensor; a first right amplifier coupled to an output of the first right optical sensor; and a second right amplifier coupled to an output of the second right optical sensor, where the normalizing the left eye electrical signal derived from the first left eye electrical signal and the second left eye electrical signal includes adjusting a gain of the first left amplifier and the second left amplifier based on a power of a left eye normalizing signal at a spectrum that is characteristic of central fixation, and where the normalizing the right eye electrical signal derived from the first right eye electrical signal and the second right eye electrical signal includes adjusting a gain of the first right amplifier and the second right amplifier based on a power of a right eye normalizing signal at a spectrum that is characteristic of central fixation. The left eye normalizing signal can include a sum of the first left eye electrical signal and the second left eye electrical signal, and the right eye normalizing signal can include a sum of the first right eye electrical signal and the second right eye electrical signal. The alignment status can include an indication of one of: the right eye and the left eye are aligned, or the right eye and the left eye are misaligned. The outputting the alignment status can include displaying the alignment status. The normalizing the left eye electrical signal can include dividing a power of the left eye electrical signal by the power of the left eye normalizing signal at the spectrum that is characteristic of central fixation, and the normalizing the right eye electrical signal can include dividing a power of the right eye electrical signal by the power of the right eye normalizing signal at the spectrum that is characteristic of central fixation. The optical scanner can be disposed to direct circularly scanned polarized light to the left retina of the subject and to the right retina of the subject. The circularly scanned polarized light can be periodically scanned at a scanning frequency, and the spectrum that is characteristic of central fixation can include at least one of: a frequency of 2.5 times the scanning frequency or a frequency of 6.5 times the scanning frequency.

According to various embodiments, a method of determining an alignment status of a subject's eyes is presented. The method includes: directing, by an optical scanner, polarized light to a left retina of the subject and to a right retina of the subject; separating, by a left polarizing beam splitter, polarized light reflected from the left retina into a first left optical signal component and a second left optical signal component; receiving, by a first left optical sensor, the first left optical signal component and generating a corresponding first left eye electrical signal; receiving, by a second left optical sensor, the second left optical signal component and generate a corresponding second left eye electrical signal; separating, by a right polarizing beam splitter, polarized light reflected from the right retina into a first right optical signal component and a second right optical signal component; receiving, by a first right optical sensor, the first right optical signal component and generating a corresponding first right eye electrical signal; receiving, by a second right optical sensor, the second right optical signal component and generating a corresponding second right eye electrical signal; normalizing a left eye electrical signal derived from the first left eye electrical signal and the second left eye electrical signal, where the normalizing the left eye electrical signal is based on a power of a left eye normalizing signal at a spectrum that is characteristic of central fixation, where a normalized left eye electrical signal is produced; normalizing a right eye electrical signal derived from the first right eye electrical signal and the second right eye electrical signal, where the normalizing the right eye electrical signal is based on a power of a right eye normalizing signal at a spectrum that is characteristic of central fixation, where a normalized right eye electrical signal is produced; determining an alignment status of the subject's eyes based on a level of the normalized left eye electrical signal and a level of the normalized right eye electrical signal; and outputting an alignment status of the subject's eyes.

Various optional features of the above embodiments include the following. The method can include directing, by a beam splitter, a portion of the polarized light reflected from the left retina and the polarized light reflected from the right retina to a normalizing optical sensor, where the normalizing optical sensor provides the left eye normalizing signal and the right eye normalizing signal. The method can include directing, by a left eye beam splitter, a portion of the polarized light reflected from the left retina to a left normalizing optical sensor, where the left normalizing optical sensor provides the left eye normalizing signal; and directing, by a right eye beam splitter, a portion of the polarized light reflected from the right retina to a right normalizing optical sensor, where the right normalizing optical sensor provides the right eye normalizing signal. The normalizing the left eye electrical signal derived from the first left eye electrical signal and the second left eye electrical signal can include adjusting a gain of a first left amplifier coupled to an output of the first optical sensor and a second left amplifier coupled to an output of the second left optical sensor based on a power of a left eye normalizing signal at a spectrum that is characteristic of central fixation, and the normalizing the right eye electrical signal derived from the first right eye electrical signal and the second right eye electrical signal can include adjusting a gain of a first right amplifier coupled to an output of the first right optical sensor and a second right amplifier coupled to an output of the second right optical sensor based on a power of a right eye normalizing signal at a spectrum that is characteristic of central fixation. The left eye normalizing signal can include a sum of the first left eye electrical signal and the second left eye electrical signal, and the right eye normalizing signal can include a sum of the first right eye electrical signal and the second right eye electrical signal. The alignment status can include an indication of one of: the right eye and the left eye are aligned, or the right eye and the left eye are misaligned. The outputting the alignment status can include displaying the alignment status. The normalizing the left eye electrical signal can include dividing a power of the left eye electrical signal by the power of the left eye normalizing signal at the spectrum that is characteristic of central fixation; and the normalizing the right eye electrical signal can include dividing a power of the right eye electrical signal by the power of the right eye normalizing signal at the spectrum that is characteristic of central fixation. The directing, by the optical scanner, polarized light to the left retina of the subject and to the right retina of the subject can include directing circularly scanned polarized light to the left retina of the subject and to the right retina of the subject. The circularly scanned polarized light can be periodically scanned at a scanning frequency, and the spectrum that is characteristic of central fixation can include at least one of: a frequency of 2.5 times the scanning frequency or a frequency of 6.5 times the scanning frequency.

Combinations, (including multiple dependent combinations) of the above-described elements and those within the specification have been contemplated by the inventors and may be made, except where otherwise indicated or where contradictory.

### BRIEF DESCRIPTION OF THE DRAWINGS

Various features of the examples can be more fully appreciated, as the same become better understood with reference to the following detailed description of the examples when considered in connection with the accompanying figures, in which:

FIG. 1 shows a computer-generated model of the effect of human Henle fiber birefringence on the polarization state of initially linearly-polarized light at 90° reflected from the fundus, measured in terms of Stokes' S₁, along with example circular scan locations for determining central fixation;

FIG. 2 is a schematic diagram of a simplified binocular RBS system;

FIG. 3 depicts RBS signals for the left eye and right eye of a test subject, illustrating a lack of RBS signal normalization;

FIG. 4 depicts RBS signals for two different test subjects, illustrating a lack of RBS signal normalization;

FIG. 5 is a schematic diagram of an RBS system with beam splitter normalization hardware according to various embodiments;

FIG. 6 is a schematic diagram of an RBS system with beam splitter normalization hardware for individual normalization for each eye according to various embodiments;

FIG. 7 is a schematic diagram of an RBS system with amplifier gain control normalization hardware according to various embodiments;

FIG. 8 is an example of a gain control curve for amplifier gain control normalization according to various embodiments;

FIG. 9 is a schematic diagram of an RBS system with RBS signal normalization according to various embodiments;

FIG. 10 is a graph of a normalized RBS signal according to various embodiments; and

FIG. 11 is a flow chart for a method of determining an alignment status of a subject's eyes according to various embodiments.

### DESCRIPTION OF THE EXAMPLES

Reference will now be made in detail to example implementations, illustrated in the accompanying drawings. Wherever convenient, the same reference numbers will be used throughout the drawings to refer to the same or like parts. In the following description, reference is made to the accompanying drawings that form a part thereof, and in which is shown by way of illustration specific exemplary examples in which the invention may be practiced. These examples are described in sufficient detail to enable those skilled in the art to practice the invention, and it is to be understood that other examples may be utilized and that changes may be made without departing from the scope of the invention. The following description is, therefore, merely exemplary.

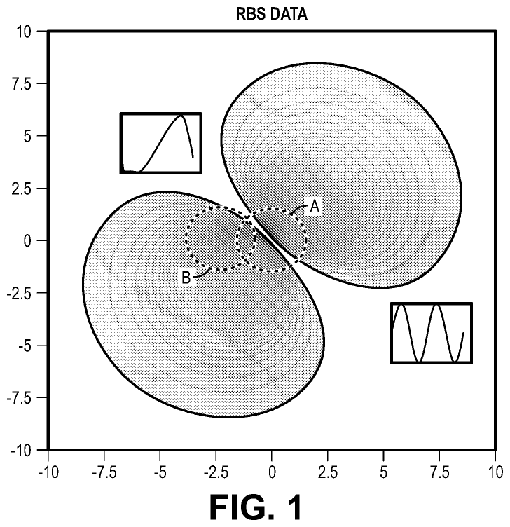

Some embodiments solve the problem of a lack of standardization of RBS signals. As shown and described herein in detail in reference to FIGS. 1, 2, 3, and 4, variability and asymmetry in both measurement devices and test subjects present challenges to using RBS signals to determine an eye alignment status, for example. Sources of such variability and asymmetry include, for example: variations in and among optical hardware, instrumental noise, instrumental asymmetry between left and right eye hardware, device-to-device variability, pupil diameter and reflectivity differences among subjects, cataract, and eye position relative to the testing device. Some embodiments solve the problems presented by such variability and asymmetry by normalizing RBS test signals. Some embodiments may perform standardized comparisons using normalized RBS test signals in order to determine, for example, an eye alignment status of a test subject. Moreover, some embodiments may be used in the context of a variety of hardware and test subject variability and asymmetry, while maintaining accurate test results.

These and other features and advantages are presented herein in reference to the figures.

In general, there are two main types of RBS systems. The first type, referred to herein as a “1f2f” system, uses simple circular scans around the presumed location of the fovea. For a 1f2f system, polarized, e.g., near-infrared, light is reflected from the foveal area in a detectable bow-tie-like pattern of polarization states (see FIG. 1), allowing localization and eye tracking. The fovea is aimed at the object of regard during fixation. When illuminated with polarized (e.g., linearly polarized, circularly polarized, elliptically polarized, etc.) near-infrared light, such as the light emitted by a low-power 785 nm or 830 nm laser diode, the uniquely arranged, radially symmetric nerve fibers (Henle fibers) surrounding the center of the fovea change the polarization state of the light being back-reflected from the underlying retinal pigment epithelium.

FIG. 1 shows a computer-generated model of the effect of human Henle fiber birefringence on the polarization state of initially linearly-polarized light at 90° reflected from the fundus, measured in terms of Stokes' S₁, along with example circular scan locations for determining central fixation. The Henle fibers are the uniquely-arranged, radially-symmetric nerve fibers surrounding the fovea. The axes are in degrees measured from the foveal center. Circular scan (counterclockwise) locations are indicated by dotted circles, and start at position 3 o'clock. As described presently, such circular scans can help establish central fixation (a), or lack thereof (b). The time signals are for a 1f2f system.

In more detail, FIG. 1 shows the distribution (Haidinger brush) of Stokes component S₁ (horizontal preference) around the center of the fovea, as a combination of a fundus image taken using polarized light and a polarizer, and a superimposed graphical printout from a computer model. The location of this “brush” can be detected by interrogating the area with a full raster scan (which is possible, but would be relatively slow), with just a few laser beam spots, or by means of a fast circular scan.

To detect central fixation, e.g., in the case of pediatric vision screeners, circular scanning followed by frequency analysis may be used. When the eye fixates on a fixation target optically at the center of the scanning circle, the returned scan signal s(t) is of a specific frequency f₂, such as twice the scanning frequency fₛ, as represented by scan (a) in FIG. 1. Alternatively, off-center fixation produces a different signal, of merely the scanning frequency for example, f₁=fₛ, as represented by scan (b) in FIG. 1. In both cases, for FIG. 1, the scanning starts at an imaginary three o'clock position on the scanning circle, shown with dotted lines, and progresses in a counter-clockwise direction. Off-center directions of gaze produce mixtures of the two frequencies and signal traces specific to the direction of gaze.

In the above described circular RBS method, the signal level is very low, because the returned light from the retina is approximately 5000 times less than the light of the scanning beam entering the eye, and is comparable with the instrumental noise. Prior art techniques have attempted to handle this problem using background subtraction (flat fielding), which slows down system performance. As an alternative, in more recent, second type, RBS systems, spatial polarization modulation was introduced, incorporating a double-pass half wave plate (HWP) spinning 9/16th as fast as the circular scan frequency fₛ. The spinning HWP works as a polarization rotator. When interacting with the Henle fibers, the rotating polarization of the incident light modulates the RBS signal and generates half multiples of the scanning frequency upon reflection. The characteristic frequencies for this system are 2.5fₛ and 6.5fₛ for central fixation, and 3.5fₛ and 5.5fₛ for off-central fixation. These half-multiple frequency signals double in amplitude and even quadruple in signal strength (fast Fourier transform (FFT) power) with 360° phase-shift subtraction, whereas most of the optical background noise (instrumental noise) at whole multiples of the scanning frequency is removed, thus eliminating the need of background subtraction, and significantly increasing the signal-to-noise ratio (SNR). This second type of RBS design is referred to herein as an spHWP system.

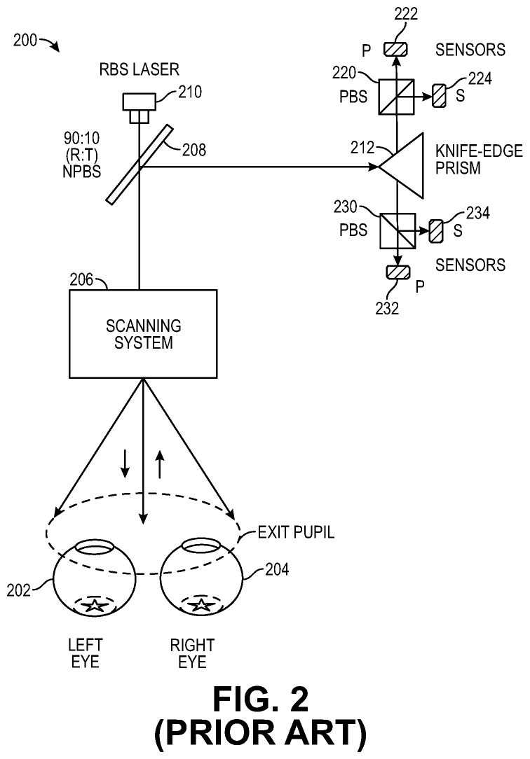

FIG. 2 is a schematic diagram of a simplified generic binocular RBS system 200. An RBS laser 210, e.g., a laser diode, provides near-infrared light to an RBS scanning system 206 through a 90:10 non-polarizing beam splitter (NPBS) 208. The RBS scanning system 206 polarizes and scans the light in a circular pattern with a frequency of fs, and directs it to the subject's left eye 202 and right eye 204. For the a spHWP RBS system, the RBS scanning system 206 incorporates a spinning HWP. A central fixation target (depicted using stars on the retinas of eyes 202, 204) optically conjugate to the plane of the scanning circle is introduced, e.g., by a beam splitter. The light reflected from the retinas of the left eye 202 and right eye 204 is redirected by the NPBS 208 to a knife-edge reflecting prism 212, which then separates the light coming from the two eyes 202, 204. For each eye 202, 204, a polarizing beamsplitter (PBS) 220, 230 decomposes, i.e., splits, the light into s- and p-components, which after measurement by corresponding sensors 222, 224, 232, 234 are used to build the Stokes vector component S₁: S₁=s−p. Not shown are waveplates, mirrors, lenses, light traps, polarization rotators, polarization compensators, scanning motor, scanning mirrors, etc.

In both types of RBS systems, central fixation is determined when the spectral power of the scanning signal returned from the retina is above a certain threshold for a characteristic frequency or combination of frequencies. This is usually done for each eye separately, and CF is determined when both eyes pass the same threshold. However, due to optical hardware asymmetries and/or the presence of certain instrumental noise (different for the signals received for the two eyes), device-to-device variability etc., applying a threshold-based decision-making may become imprecise and produce erroneous results. Furthermore, pupil diameter and retinal reflectivity vary from subject to subject. Finally, the position of the eye in the exit pupil of the device can also affect the signal amplitude. Some embodiments address all of the above sources of variability and asymmetry through the use of normalization.

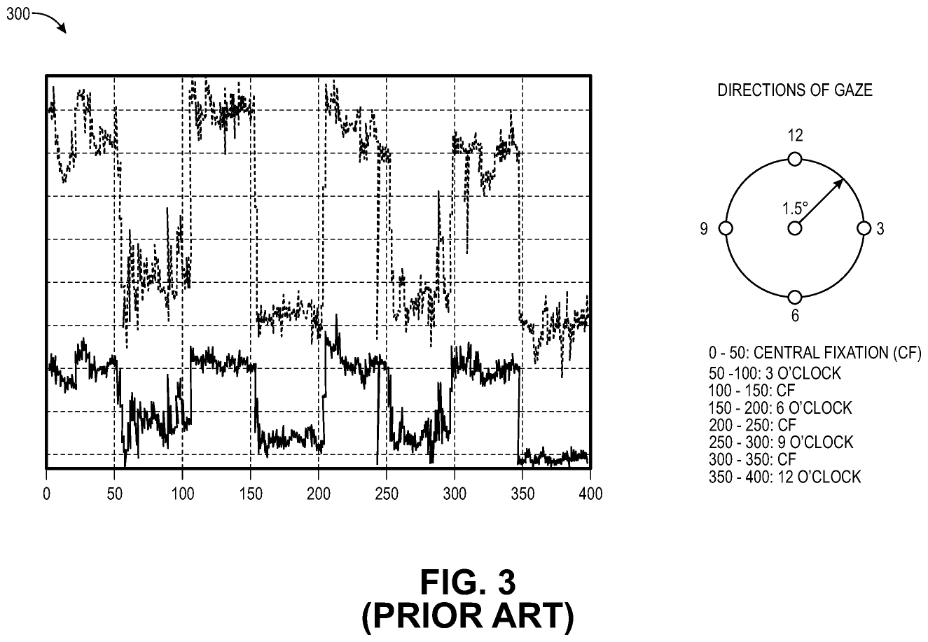

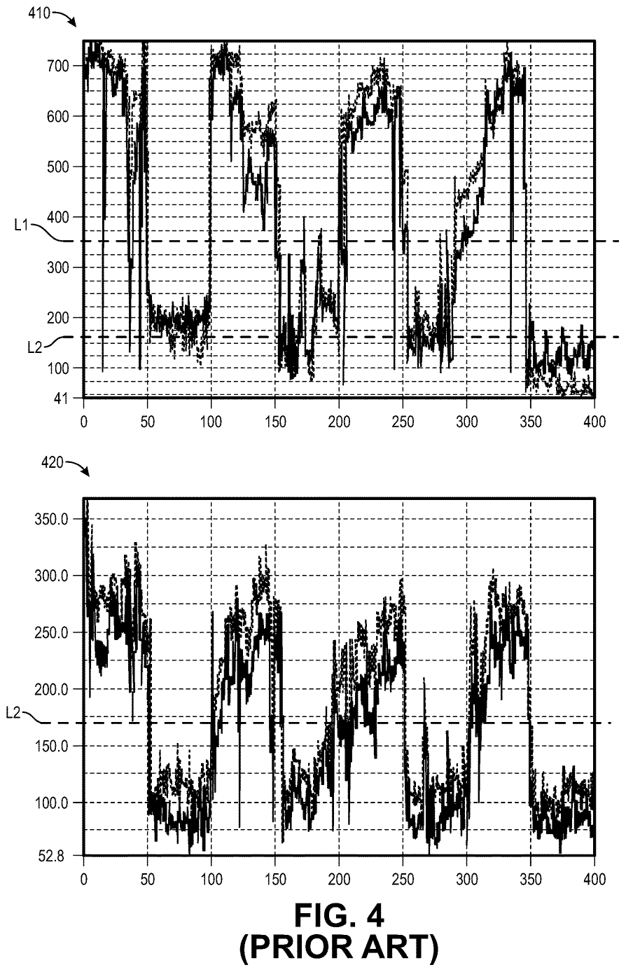

The problem of using a single threshold for both eyes is illustrated below in reference to FIG. 3, and the problem with using a single threshold for multiple subjects is illustrated below in reference to FIG. 4.

FIG. 3 depicts RBS signals for the left eye and right eye of a test subject, illustrating a lack of RBS signal normalization. The RBS signals represented by chart 300 of FIG. 3 are the second Stokes vector component S1, or s-p. Spectral powers at CF-characteristic frequencies over time are depicted, while the test subject was fixating on a central target during acquisitions 0-50, 100-150, 200-250, and 300-350. At all other times the subject was fixating on points 1.5° away from the central target (off-CF). The traces are unfiltered and are from an spHWP system. The top trace represents the subject's right eye, and the bottom trace represents the subject's left eye. Each trace represents the combined power P25+P65 (for CF-characteristic frequencies 2.5fₛ and 6.5fₛ, respectively), over a period of ~80 s (400 acquisitions). The spectra were obtained from component S₁ of the Stokes vector. Each data point was derived from the FFT obtained from a time-domain RBS signal acquisition of duration 200 ms.

As is apparent from FIG. 3, while the test subject's vision was normal and he was responding adequately to instructions to change the direction of gaze, the traces are different, most likely because the two channels have different gain and bias, or are in a different position in the exit pupil of the device. Some pupil size asymmetry, or even cataract, could also have been the cause. This would prevent the software designer from using simple thresholds applied naïvely to the power spectrum. For example, no single threshold separates CF from non-CF for both eyes.

FIG. 4 depicts RBS signals for two different test subjects, in charts 410 and 420, illustrating a lack of RBS signal normalization. The same RBS system was employed for data collection, based on Stokes component S₁, and the same data acquisition protocol was used, as the one for FIG. 3. Yet, this time data were recorded from two different human test subjects. Again, CF-characteristic spectral power P25+P65 was calculated over a period of ~80 s (400 acquisitions), once for each acquisition epoch of approximately 200 ms duration. The subjects were fixating on a target during acquisitions 0-50, 100-150, 200-250, and 300-350. At all other times the subjects were fixating on points 1.5° away from the central target (off-CF). The spectral traces are unfiltered and are from an spHWP system.

It can immediately be seen by comparing charts 410 and 420 of FIG. 4, that although there is good symmetry between the eyes for each subject (unlike what is shown in FIG. 3), the spectral power for the subject represented by chart 410 is more than twice higher than for the subject represented by chart 420. This presents a problem, because a discrimination threshold at level L1 (about 350 FFT units), which would work well for the subject represented by chart 410, would not work at all for the subject represented by chart 420. (Please note the different vertical scale in the two plots). Vice versa, discrimination level L2 (about 170 FFT units), appropriate for the subject represented by chart 420 would fail completely for the subject represented by chart 410. Some embodiments solve this problem through the use of normalization, which can render the power traces from different subjects, or from one and the same subject under different conditions, comparable-such that a universal threshold for central fixation may be applied.

An attempt to utilize normalization is disclosed in U.S. Pat. No. 9,713,423. There, the spectral power P45 (at 4.5f) is used for normalization of P25+P65. The spectral power P45 is widely independent of the direction of gaze, that is, it is not CF-characteristic, which is why it was employed as a normalizing quantity. However, depending on a number of factors, it may not necessarily change with pupil size, retinal reflectivity, or position of the eye within the exit pupil in the same way as (P25+P65) does. Also, in many optical designs, P45 as a function of corneal retardance and corneal azimuth (CR, CA), is not a flat distribution (as shown by computer modeling). Another problem is that some 4.5f components can penetrate into the channels as back-reflection instrumental noise, inseparable from the P45 coming from the retina, which can adversely impact precision, causing erroneous results. Further, neutralization using the 4.5f signal only works with a spHWP, which is more complicated than a 1f2f system (one with no polarization rotator).

According to various embodiments, several normalization techniques are presented. An (s-p) signal, or CF-characteristic power spectrum thereof (e.g., P25+P65 for spHWP or P20 for 1f2f) may be normalized. The normalization may be based on a normalization signal, e.g., an (s+p) signal, or a CF-characteristic power spectrum thereof (e.g., P25+P65 for spHWP or P20 for 1f2f). For example, for a spHWP RBS system, the normalized signals may be the 2.5f and 6.5f frequencies of (s-p) for each eye, and the normalization signal may be the amplitudes of the FFT spectral peaks (P25+P65) obtained from the (time) signal (s+p).

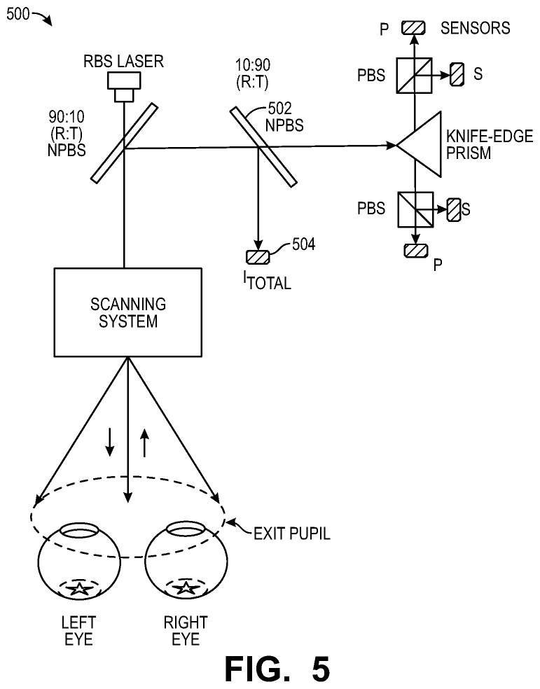

The normalization techniques disclosed herein solve the problems of variability and asymmetry in both measurement devices and test subjects, and also solve the problems presented by prior art attempts at normalization. Such superior normalization techniques are shown and described presently in reference to FIGS. 5-11.

FIG. 5 is a schematic diagram of an RBS system 500 with beam splitter normalization hardware according to various embodiments. The RBS system 500 with beam splitter normalization hardware is similar to the generic RBS system as shown and described herein in reference to FIG. 2, except that it includes a 10:90 NPBS 502 before the knife-edge prism that redirects a portion of the light reflected from the subject's retinas to a normalization sensor 504. The normalization signal acquired by the normalization sensor 504 is Iₜₒₜₐₗ=S₀, (or a proportional part of it) and is the same for both eyes. To avoid unnecessary loss of light returned from the eye (potentially resulting in a deteriorated signal-to-noise ratio), the NPBS 502 may pass a large portion of the light towards the knife-edge prism (as show in FIG. 5, 90% transmission), and reflect a smaller portion towards the normalization sensor 504 (here 10% reflection). The quantity Iₜₒₜₐₗ, equivalent to Stokes element S₀=s+p, or a known proportional part of it, is proportional to the total amount of light returning to the sensors and is polarization independent. It can be used for normalization, and accounts for changes in the pupil size, retinal reflectivity, or even cataract.

To use the Iₜₒₜₐₗ signal for normalization, power spectra for one or more frequencies characteristic of central fixation may be extracted from the signal, e.g., using an FFT. The power CF-characteristic power spectrum (e.g., P25+P65 for spHWP or P20 for 1f2f) of the RBS signal (e.g., S₁) may be divided by the CF-characteristic power spectrum (e.g., P25+P65 for spHWP or P20 for 1f2f) of the normalization signal (e.g., Iₜₒₜₐₗ). The resulting normalized signal may be compared with a threshold to determine central fixation of the subject's eyes.

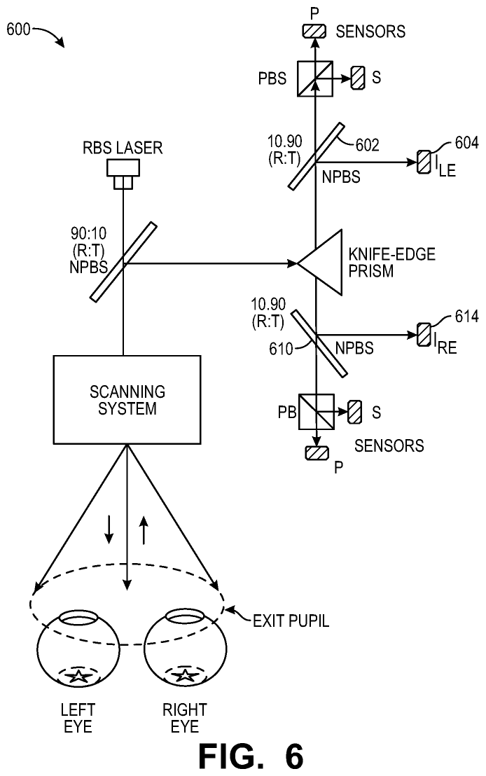

FIG. 6 is a schematic diagram of an RBS system 600 with beam splitter normalization hardware for individual normalization for each eye according to various embodiments. The RBS system 600 with beam splitter normalization hardware for individual normalization for each eye is similar to the generic RBS system as shown and described herein in reference to FIG. 2, except that it includes a 10:90 NPBS 602 between the knife-edge prism and the sensors for the left eye signals that redirects a portion of the light reflected from the subject's left retina to a left eye signal normalization sensor 604, and includes a 10:90 NPBS 612 between the knife-edge prism and the sensors for the right eye signals that redirects a portion of the light reflected from the subject's right retina to a right eye signal normalization sensor 614. To avoid unnecessary loss of light returned from the eye (potentially resulting in a deteriorated signal-to-noise ratio) and passed to the left and right eye sensors, the NPBS 602, 612 may pass a large portion of the light towards the left and right eye sensors (as show in FIG. 6, 90% transmission), and reflect a smaller portion towards the normalization sensors 604, 614 (as shown, 10% reflection).

The quantity I_{LE}, equivalent to Stokes element S₀ for the left eye signal or a known proportional part of it, is proportional to the total amount of light returning to the left eye sensors and is polarization independent. It can be used for normalization of the left eye RBS signal, and accounts for changes in the pupil size, retinal reflectivity, or even cataract. Likewise, the quantity IRE, equivalent to Stokes element S₀ for the right eye signal or a known proportional part of it, is proportional to the total amount of light returning to the right eye sensors and is polarization independent. It can be used for normalization of the right eye RBS signal, and accounts for changes in the pupil size, retinal reflectivity, or even cataract.

To use the I_{LE} signal for normalization of the left eye RBS signal, power spectra for one or more frequencies characteristic of central fixation may be extracted from the signal, e.g., using an FFT. The power CF-characteristic power spectrum (e.g., P25+P65 for spHWP or P20 for 1f2f) of the left eye RBS signal (e.g., S₁ for the left eye) may be divided by the CF-characteristic power spectrum (e.g., P25+P65 for spHWP or P20 for 1f2f) of the left eye normalization signal (e.g., I_{LE}). The resulting normalized left eye signal may be compared with a threshold to determine central fixation of the subject's left eye.

The analogous procedure applies for using the IRE signal for normalization of the right eye RBS signal. Namely, power spectra for one or more frequencies characteristic of central fixation may be extracted from the signal, e.g., using an FFT. The power CF-characteristic power spectrum (e.g., P25+P65 for spHWP or P20 for 1f2f) of the right eye RBS signal (e.g., S₁ for the right eye) may be divided by the CF-characteristic power spectrum (e.g., P25+P65 for spHWP or P20 for 1f2f) of the right eye normalization signal (e.g., IRE). The resulting normalized right eye signal may be compared with a threshold to determine central fixation of the subject's right eye. The threshold used for the right eye signal may be the same threshold as is used for the left eye signal.

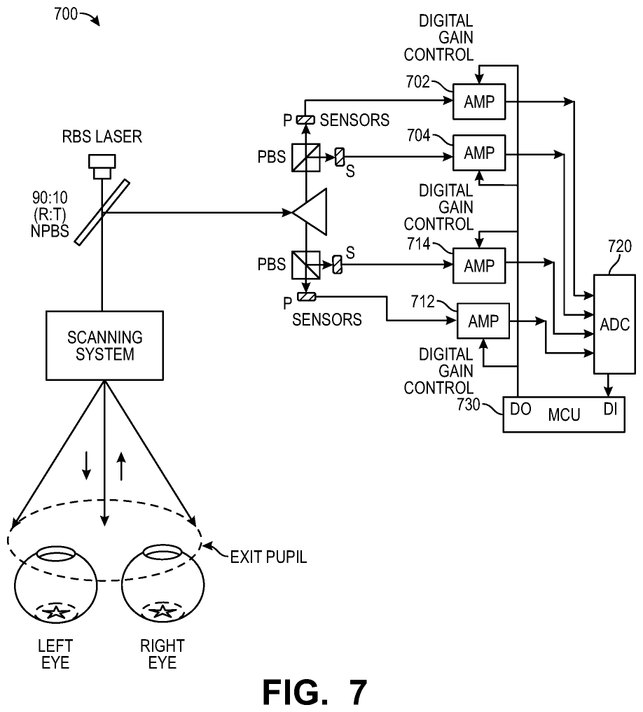

FIG. 7 is a schematic diagram of an RBS system 700 with amplifier gain control normalization hardware according to various embodiments. The system 700 is similar to the generic RBS system 200 as shown and described in reference to FIG. 2, with the following differences. The system 700 includes amplifiers 702, 704, 712, 714 with digital gain control. The amplifiers 704, 702 control the amplitude of the individual left eye polarization signals s and p, respectively, and the amplifiers 714, 712 control the amplitude of the individual right eye polarization signals s and p, respectively. In system 700, a microcontroller unit (MCU) 730, or any other CPU, communicates with the amplifiers 702, 704, 712, 714 over digital output lines DO. One or both of the digital output lines and an analog-to-digital converter (ADC) 720 may be integrated in the MCU 730, or may be separate as shown.

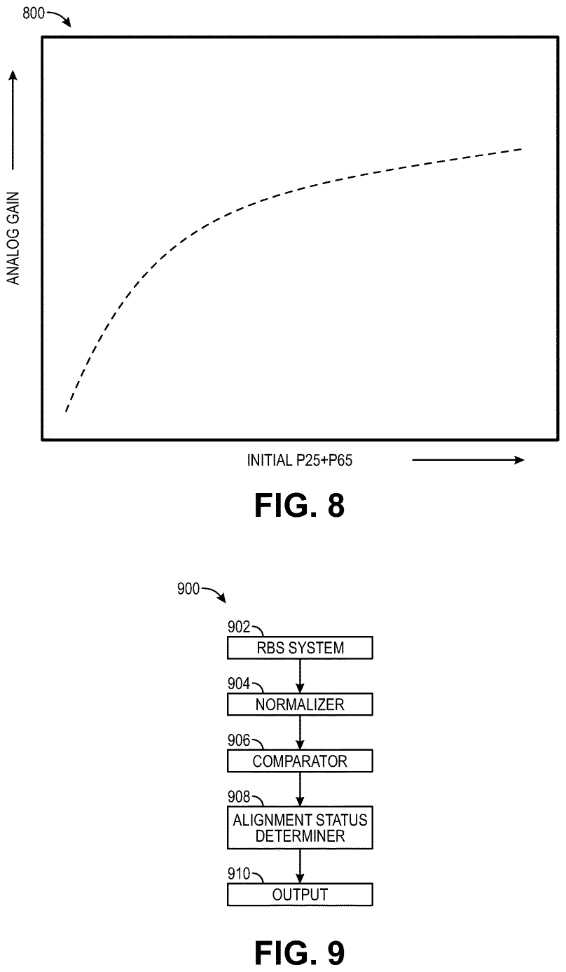

Embodiments according to the RBS system 700 achieve gain of the analog signals coming from the four sensors that brings the P25+P65 power (for spHWP, by way of non-limiting example) into a normal range, where a standard discriminating threshold can be applied (CF vs off-CF). After a short measurement period (one to several seconds), spectral analysis is performed, e.g., of the S₀=(s+p) signal, and the analog gain is set to a new value according to a calibration curve that may be nonlinear, e.g., as shown in FIG. 8, e.g., a square-root function. Gains are adjusted as described in order to obtain new levels of P25+P65 power (respectively, P20 power for 1f2f) that match a previously chosen standard target value. Subsequently, the individual signals for the Stokes S₁ component (s-p) for each eye are formed from the outputs of the amplifiers 702, 704, 712, 714, and compared to a universal threshold to determine central fixation of each eye.

Note that according to various embodiments, the system 700 may be modified by using amplifiers in place of amplifiers 702, 704, 712, 714 that are controlled by analog voltage or current. According to such embodiments, the controlling voltage (or current) may be supplied by the MCU 730 over a digital-to-analog converter, which can be integral with the MCU 730 or separate.

FIG. 8 is an example of a gain control curve 800 for amplifier gain control normalization according to various embodiments. The gain control curve may be used to implement embodiments of the RBS system 700 as shown and described in reference to FIG. 7. The gain control curve may determine the amount of gain of amplifiers 702, 704, 712, 714 as a function of, that is, as determined by, the power of the initial P25+P65 (or P20, for 1f2f) (s+p) signal from such amplifiers. The x-axis represents the initial (measured) P25+P65 (or P20, for 1f2f) value of the (s+p) signal, and the y-axis represents the amount gain directed to the amplifiers. According to some embodiments, the gain control curve may represent the square root function, y=\sqrt{x}.

FIG. 9 is a schematic diagram of an RBS system 900 with RBS signal normalization according to various embodiments. The normalization in RBS system 900 may be implemented using software and/or in hardware, according to various embodiments.

System 900 includes an RBS system 902, which may be implemented as shown and described herein in reference to FIG. 2. The RBS system 902 according to system 900 may be an spHWP system, or a 1f/2f system with no polarization rotator. System 900 may be used with either type of RBS system. By way of non-limiting example, the system 900 is described in reference to a spHWP RBS system. However, the system 900 may be implemented for a 1f2f RBS system, e.g., by using P20 instead of P20+P65 as disclosed herein. The RBS system 902 produces output signals, e.g., an s signal for the left eye, a p signal for the left eye, an s signal for the right eye, and a p signal for the right eye. These signals are passed to the normalizer via a communicative coupling, e.g., direct electrical connections between RBS system 900 and normalizer 904.

According to some embodiments, the normalizer 904 may be implemented using software and a processor, such as a general purpose CPU or a special purpose processor, such as an FPGA or MCU. The normalizer may build an (s+p) normalization signal that is close to the total power using digitized measured s- and p-polarization components (respectively, Fresnel's field amplitudes E²_{0y} and E²₀ₓ). In other words, the processor may construct S₁=(s−p) from the digitized s- and p-analog signals and calculate the P25 and P65 spectral components thereof, to obtain (P25+P65) (s-p). This may be performed for both eyes separately. The processor may also construct S₀=(s+p) from the same s- and p-digitized signals and calculate the P25 and P65 spectral components thereof, to obtain (P25+P65) (s+p). This may be performed for both eyes in combination, or for each eye separately. This signal, or signals, is/are then used to normalize the (P25+P65)₍ₛ₋ₚ₎ signal (P25+P65)ₙₒᵣₘ for each eye as follows, by way of non-limiting example:

$${( P 25 + P 65 )}_{norm} = \frac{{( P 25 + P 65 )}_{( s - p )}}{{( P 25 + P 65 )}_{( s + p )}}$$

According to some embodiments, the normalizer 904 may be implemented in dedicated hardware that performs certain operations in the analog signal domain. According to some embodiments, one or both of the quantities (s+p) and (s−p) are built in hardware, which performs the sum and/or difference in analog, e.g., for each eye. According to some embodiments, the analog signals representing one or both of (s+p) and (s−p), e.g., for each eye, are digitized, and the quantity (s−p)/(s+p) is computed in the digital domain for each eye, e.g., using a programmed processor as described above. According to some embodiments, the quantity (s−p)/(s+p) is computed in the analog domain for each eye, e.g., using one or more analog dividers, and then the result is digitized, obtaining a normalized signal, e.g., for each eye. Once the digitized signal for (s−p)/(s+p) is obtained for each eye according to the various embodiments, spectral analysis is performed, e.g., using a programmed processor, to obtain the normalized power spectrum at spectra characteristic of central fixation, e.g., P25+P65, for each eye. This represents another possible way to compute the normalized signal (P25+P65)ₙₒᵣₘ for each eye according to various embodiments.

Whether computed within the digital domain, the analog domain, or a combination thereof, and whether determined using software, hardware, or a combination thereof, the normalized signals characteristic of central fixation for each eye are passed from the normalizer 904 to a comparator 906. The signal or signals may be passed on electrical wires, for example, that communicatively couple the normalizer 904 to the comparator 906.

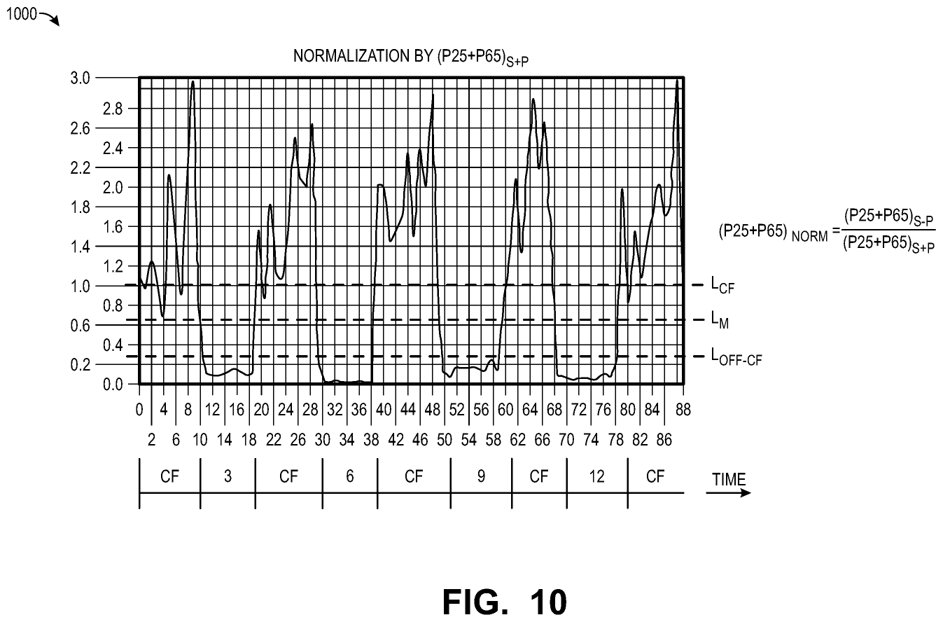

The comparator 906 compares the normalized signal for each eye to a threshold. Example suitable threshold values are shown and described herein in reference to FIG. 10, for example. The comparator 906 determines whether the normalized signal, for each eye, is at least as great as the threshold. The comparator thus produces signals for each eye that indicate whether or not the respective eye is centrally fixated. The comparator communicates these signals to the alignment status determiner 908, e.g., via electrical wires that communicatively couple the comparator 906 to the alignment status determiner.

The alignment status determiner 908, which may be implemented by a programmed processor as disclosed herein, associates an alignment status to the combination of signals that it receives from the comparator 906. Possible alignment statuses include: neither eye centrally fixated, at least one eye not centrally fixated, one eye centrally fixated, left eye centrally fixated, right eye centrally fixated, both eyes centrally fixated. The alignment status determiner 908 may thus associate an alignment status to the combination of signals it receives from the comparator 906. The alignment status determiner 908 passes the alignment status to the output 910, e.g., via electrical wires in digital form.

The output 910 may take any of a variety of forms. According to some embodiments, the output 910 includes a computer screen, which visually displays the alignment status. According to some embodiments, the output 910 provides the alignment status to another device, such as a computer, e.g., a medical records computer. Any of a variety of other implementations of output 910 may be used in addition or in the alternative.

FIG. 10 is a graph 1000 of a normalized RBS signal according to various embodiments. In particular, the graph 1000 shows normalization by (s+p) applied to the CF-relevant composite power (P25+P65) of a spHWP system according to an implementation of the system 900 as shown and described herein in reference to FIG. 9. The test subject was asked to fixate on a central target, and on four peripheral targets, as in the previous examples. The normalized signal (P25+P65)ₙₒᵣₘ was consistently above threshold L_{CF}=1.0 during central fixation, and below L_{off-CF}=0.3 during fixation on off-center points. Various embodiments may employ threshold values within this range, by way of non-limiting example. For example, a possible threshold value may be be Lₘ=0.65.

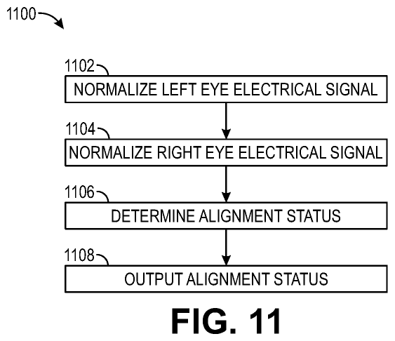

FIG. 11 is a flow chart for a method 1100 of determining an alignment status of a subject's eyes according to various embodiments. The method 1100 may be performed by any of the systems shown and described herein, e.g., system 500, 600, 700, or 900, or by any combination thereof. The method 1100 may be used with a spHWP or 1f2f RBS system.

At 1102, the method 1100 normalizes a left eye electrical signal that provides an indication of central fixation of the subject's left eye. The left eye electrical signal may be provided by an RBS system. The left eye electrical signal may be normalized using a power spectrum of a normalization signal at one or more frequencies characteristic of central fixation as disclosed herein, e.g., in reference to any of FIGS. 5, 6, 7, and/or 9.

At 1104, the method 1100 normalizes a right eye electrical signal that provides an indication of central fixation of the subject's right eye. The right eye electrical signal may be provided by an RBS system. The right eye electrical signal may be normalized using a power spectrum of a normalization signal at one or more frequencies characteristic of central fixation as disclosed herein, e.g., in reference to any of FIGS. 5, 6, 7, and/or 9.

At 1106, an alignment status of the subject is determined. Possible alignment statuses include: neither eye centrally fixated, at least one eye not centrally fixated, one eye centrally fixated, left eye centrally fixated, right eye centrally fixated, both eyes centrally fixated. An alignment status from among any combination of these alignment statuses may be determined according to various embodiments, e.g., by comparing the normalized left eye electrical signal and/or the normalized right eye electrical signal to a threshold value. The threshold value may be any of a variety of values, which may be determined according to the particular implementation. A single threshold value may be utilized for both eyes of a subject, and for multiple subjects, due to the normalization process disclosed herein.

At 1108, the alignment status is output. The output may be in the form of a display, e.g., on a computer monitor or specialized device, or may be provided to a separate computer or device, such as a medical records computer.

Note that the particular CF-characteristic frequencies disclosed herein (e.g., 2.0f, 2.5f, 6.5f) are non-limiting. For example, for spHWP RBS systems, different modulation frequencies, such as 7/8f or 11/8f, for a single-pass spinning HWP, rather than 9/8f, will yield different CF frequencies from 2.5f and 6.5f. Further, although for a spHWP system that utilizes a double-pass spinning HWP, the spinning HWP frequency of 9/16f yields 2.5f and 6.5f for the centration frequencies, for other frequencies of the spinning HWP, different CF frequencies are generated. In general, the particular CF-characteristic frequency or frequencies may depend on the spinning HWP modulation frequency and/or other parameters of the RBS system.

Thus, systems for, and methods of, normalization that overcome dependence on changes in the pupil size, retinal reflectivity, position within the exit pupil of the device, or even cataract are presented herein. The disclosed system and methods are straightforward to implement and do not need high-precision or repetitive adjustment. Also, the systems and method may be used with spHWP RBS systems, or with the simpler 1f2f RBS systems.

Certain examples can be performed using a computer program or set of programs. The computer programs can exist in a variety of forms both active and inactive. For example, the computer programs can exist as software program(s) comprised of program instructions in source code, object code, executable code or other formats; firmware program(s), or hardware description language (HDL) files. Any of the above can be embodied on a transitory or non-transitory computer readable medium, which include storage devices and signals, in compressed or uncompressed form. Exemplary computer readable storage devices include conventional computer system RAM (random access memory), ROM (read-only memory), EPROM (erasable, programmable ROM), EEPROM (electrically erasable, programmable ROM), and magnetic or optical disks or tapes.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented using computer readable program instructions that are executed by a processor.

These computer readable program instructions may be provided to a processor of a general-purpose computer, special purpose computer, or other programmable data processing apparatus, to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

In embodiments, the computer readable program instructions may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the C programming language or similar programming languages. The computer readable program instructions may execute entirely on a user's computer, partly on the user's computer, as a stand-alone software package, partly on the user's computer and partly on a remote computer or entirely on the remote computer or server.

As used herein, the terms “A or B” and “A and/or B” are intended to encompass A, B, or {A and B}. Further, the terms “A, B, or C” and “A, B, and/or C” are intended to encompass single items, pairs of items, or all items, that is, all of: A, B, C, {A and B}, {A and C}, {B and C}, and {A and B and C}. The term “or” as used herein means “and/or.”

As used herein, language such as “at least one of X, Y, and Z,” “at least one of X, Y, or Z,” “at least one or more of X, Y, and Z,” “at least one or more of X, Y, or Z,” “at least one or more of X, Y, and/or Z,” or “at least one of X, Y, and/or Z,” is intended to be inclusive of both a single item (e.g., just X, or just Y, or just Z) and multiple items (e.g., {X and Y}, {X and Z}, {Y and Z}, or {X, Y, and Z}). The phrase “at least one of” and similar phrases are not intended to convey a requirement that each possible item must be present, although each possible item may be present.

The techniques presented and claimed herein are referenced and applied to material objects and concrete examples of a practical nature that demonstrably improve the present technical field and, as such, are not abstract, intangible or purely theoretical. Further, if any claims appended to the end of this specification contain one or more elements designated as “means for [perform]ing [a function] . . . ” or “step for [perform]ing [a function] . . . ”, it is intended that such elements are to be interpreted under 35 U.S.C. § 112 (f). However, for any claims containing elements designated in any other manner, it is intended that such elements are not to be interpreted under 35 U.S.C. § 112(f).

While the invention has been described with reference to the exemplary examples thereof, those skilled in the art will be able to make various modifications to the described examples without departing from the true spirit and scope. The terms and descriptions used herein are set forth by way of illustration only and are not meant as limitations. In particular, although the method has been described by examples, the steps of the method can be performed in a different order than illustrated or simultaneously. Those skilled in the art will recognize that these and other variations are possible within the spirit and scope as defined in the following claims and their equivalents.

What is claimed is:

1. A system for determining an alignment status of a subject's eyes, the system comprising:
an optical scanner disposed to direct polarized light to a left retina of the subject and to a right retina of the subject;
a left polarizing beam splitter disposed to separate polarized light reflected from the left retina into a first left optical signal component and a second left optical signal component;
a first left optical sensor disposed to receive the first left optical signal component and generate a corresponding first left eye electrical signal;
a second left optical sensor disposed to receive the second left optical signal component and generate a corresponding second left eye electrical signal;
a right polarizing beam splitter disposed to separate polarized light reflected from the right retina into a first right optical signal component and a second right optical signal component;
a first right optical sensor disposed to receive the first right optical signal component and generate a corresponding first right eye electrical signal;
a second right optical sensor disposed to receive the second right optical signal component and generate a corresponding second right eye electrical signal;
an electronic processor; and
persistent electronic memory comprising instructions that, when executed by the electronic processor, configure the electronic processor to perform actions comprising:
normalizing a left eye electrical signal derived from the first left eye electrical signal and the second left eye electrical signal, wherein the normalizing the left eye electrical signal is based on a power of a left eye normalizing signal at a spectrum that is characteristic of central fixation, wherein a normalized left eye electrical signal is produced;
normalizing a right eye electrical signal derived from the first right eye electrical signal and the second right eye electrical signal, wherein the normalizing the right eye electrical signal is based on a power of a right eye normalizing signal at a spectrum that is characteristic of central fixation, wherein a normalized right eye electrical signal is produced;
determining an alignment status of the subject's eyes based on a level of the normalized left eye electrical signal and a level of the normalized right eye electrical signal; and
outputting an alignment status of the subject's eyes.

2. The system of claim 1, further comprising:
a beam splitter disposed to direct a portion of the polarized light reflected from the left retina and the polarized light reflected from the right retina to a normalizing optical sensor,
wherein the normalizing optical sensor provides the left eye normalizing signal and the right eye normalizing signal.

3. The system of claim 1, further comprising:
a left eye beam splitter disposed to direct a portion of the polarized light reflected from the left retina to a left normalizing optical sensor,
wherein the left normalizing optical sensor provides the left eye normalizing signal; and
a right eye beam splitter disposed to direct a portion of the polarized light reflected from the right retina to a right normalizing optical sensor,
wherein the right normalizing optical sensor provides the right eye normalizing signal.

4. The system of claim 1, further comprising:
a first left amplifier coupled to an output of the first left optical sensor;
a second left amplifier coupled to an output of the second left optical sensor;
a first right amplifier coupled to an output of the first right optical sensor; and
a second right amplifier coupled to an output of the second right optical sensor;
wherein the normalizing the left eye electrical signal derived from the first left eye electrical signal and the second left eye electrical signal comprises adjusting a gain of the first left amplifier and the second left amplifier based on a power of a left eye normalizing signal at a spectrum that is characteristic of central fixation, and
wherein the normalizing the right eye electrical signal derived from the first right eye electrical signal and the second right eye electrical signal comprises adjusting a gain of the first right amplifier and the second right amplifier based on a power of a right eye normalizing signal at a spectrum that is characteristic of central fixation.

5. The system of claim 1, wherein:
the left eye normalizing signal comprises a sum of the first left eye electrical signal and the second left eye electrical signal, and
the right eye normalizing signal comprises a sum of the first right eye electrical signal and the second right eye electrical signal.

6. The system of claim 1, wherein the alignment status comprises an indication of one of: the right eye and the left eye are aligned, or the right eye and the left eye are misaligned.

7. The system of claim 1, wherein the outputting the alignment status comprises displaying the alignment status.

8. The system of claim 1, wherein:
the normalizing the left eye electrical signal comprises dividing a power of the left eye electrical signal by the power of the left eye normalizing signal at the spectrum that is characteristic of central fixation, and
the normalizing the right eye electrical signal comprises dividing a power of the right eye electrical signal by the power of the right eye normalizing signal at the spectrum that is characteristic of central fixation.

9. The system of claim 1, wherein the optical scanner is disposed to direct circularly scanned polarized light to the left retina of the subject and to the right retina of the subject.

10. The system of claim 9, wherein the circularly scanned polarized light is periodically scanned at a scanning frequency, and wherein the spectrum that is characteristic of central fixation comprises at least one of: a frequency of 2.5 times the scanning frequency or a frequency of 6.5 times the scanning frequency.

11. A method of determining an alignment status of a subject's eyes, the method comprising:
directing, by an optical scanner, polarized light to a left retina of the subject and to a right retina of the subject;
separating, by a left polarizing beam splitter, polarized light reflected from the left retina into a first left optical signal component and a second left optical signal component;
receiving, by a first left optical sensor, the first left optical signal component and generating a corresponding first left eye electrical signal;
receiving, by a second left optical sensor, the second left optical signal component and generate a corresponding second left eye electrical signal;
separating, by a right polarizing beam splitter, polarized light reflected from the right retina into a first right optical signal component and a second right optical signal component;
receiving, by a first right optical sensor, the first right optical signal component and generating a corresponding first right eye electrical signal;
receiving, by a second right optical sensor, the second right optical signal component and generating a corresponding second right eye electrical signal;
normalizing a left eye electrical signal derived from the first left eye electrical signal and the second left eye electrical signal, wherein the normalizing the left eye electrical signal is based on a power of a left eye normalizing signal at a spectrum that is characteristic of central fixation, wherein a normalized left eye electrical signal is produced;
normalizing a right eye electrical signal derived from the first right eye electrical signal and the second right eye electrical signal, wherein the normalizing the right eye electrical signal is based on a power of a right eye normalizing signal at a spectrum that is characteristic of central fixation, wherein a normalized right eye electrical signal is produced;
determining an alignment status of the subject's eyes based on a level of the normalized left eye electrical signal and a level of the normalized right eye electrical signal; and
outputting an alignment status of the subject's eyes.

12. The method of claim 11, further comprising:
directing, by a beam splitter, a portion of the polarized light reflected from the left retina and the polarized light reflected from the right retina to a normalizing optical sensor, wherein the normalizing optical sensor provides the left eye normalizing signal and the right eye normalizing signal.

13. The method of claim 11, further comprising:
directing, by a left eye beam splitter, a portion of the polarized light reflected from the left retina to a left normalizing optical sensor, wherein the left normalizing optical sensor provides the left eye normalizing signal; and
directing, by a right eye beam splitter, a portion of the polarized light reflected from the right retina to a right normalizing optical sensor, wherein the right normalizing optical sensor provides the right eye normalizing signal.

14. The method of claim 11,
wherein the normalizing the left eye electrical signal derived from the first left eye electrical signal and the second left eye electrical signal comprises adjusting a gain of a first left amplifier coupled to an output of the first optical sensor and a second left amplifier coupled to an output of the second left optical sensor based on a power of a left eye normalizing signal at a spectrum that is characteristic of central fixation, and
wherein the normalizing the right eye electrical signal derived from the first right eye electrical signal and the second right eye electrical signal comprises adjusting a gain of a first right amplifier coupled to an output of the first right optical sensor and a second right amplifier coupled to an output of the second right optical sensor based on a power of a right eye normalizing signal at a spectrum that is characteristic of central fixation.

15. The method of claim 11, wherein:
the left eye normalizing signal comprises a sum of the first left eye electrical signal and the second left eye electrical signal, and
the right eye normalizing signal comprises a sum of the first right eye electrical signal and the second right eye electrical signal.

16. The method of claim 11, wherein the alignment status comprises an indication of one of: the right eye and the left eye are aligned, or the right eye and the left eye are misaligned.

17. The method of claim 11, wherein the outputting the alignment status comprises displaying the alignment status.

18. The method of claim 11, wherein:
the normalizing the left eye electrical signal comprises dividing a power of the left eye electrical signal by the power of the left eye normalizing signal at the spectrum that is characteristic of central fixation; and
the normalizing the right eye electrical signal comprises dividing a power of the right eye electrical signal by the power of the right eye normalizing signal at the spectrum that is characteristic of central fixation.

19. The method of claim 11, wherein the directing, by the optical scanner, polarized light to the left retina of the subject and to the right retina of the subject comprises directing circularly scanned polarized light to the left retina of the subject and to the right retina of the subject.

20. The method of claim 19, wherein the circularly scanned polarized light is periodically scanned at a scanning frequency, and wherein the spectrum that is characteristic of central fixation comprises at least one of: a frequency of 2.5 times the scanning frequency or a frequency of 6.5 times the scanning frequency.
